(19) United States
(12) Patent Application Publication
(10) Pub. No.: US 2025/0073439 A1
(43) Pub. Date: Mar. 6, 2025

(54) SYSTEMS AND METHODS FOR TREATING LIPS AND SKIN

(71) Applicant: John H. SHADDUCK, Menlo Park, CA (US)

(72) Inventor: John H. SHADDUCK, Menlo Park, CA (US)

(21) Appl. No.: 18/821,028

(22) Filed: Aug. 30, 2024

Related U.S. Application Data
(60) Provisional application No. 63/535,896, filed on Aug. 31, 2023.

(51) Int. Cl. A61M 37/00

(52) U.S. Cl. CPC A61M 37/0092; A61M 2205/0294; A61M 2210/04; A61M 2210/0625

(57) ABSTRACT

Devices and methods for treating a skin of a patient using a serum comprising a plurality of microbubbles and a plurality of active elements are disclosed herein. The system can comprise a handpiece comprising a proximal end and a distal end; a distal tip at the distal end of the handpiece, the distal tip configured to deliver the serum; an ultrasonic transducer in the distal tip; and an inflow port in the distal tip in fluid communication with a source configured to hold the serum, where the inflow port is configured for topical application to the skin; wherein application of acoustic energy from the ultrasonic transducer causes the plurality of microbubbles in the serum delivered to the skin to oscillate causing cavitation of the plurality of microbubbles causing permeabilization of the skin allowing for penetration of the plurality of active elements in the serum to penetrate the skin.

### CROSS-REFERENCE TO RELATED APPLICATIONS

This application claims priority U.S. Provisional Patent Application No. 63/535,896 filed Aug. 31, 2023, which is incorporated herein by reference in its entirety.

### BACKGROUND

The present invention relates to ultrasound devices for treating a subject's lips or skin and, more particularly, to devices that use acoustic energy to cause cavitation in serums to enhance penetration of micro-vesicle serums into tissue.

### SUMMARY OF THE INVENTION

The systems and methods corresponding to the invention relate in general to a combination system for use in the fields of lip care, skincare, and hair restoration, wherein the combination includes (i) an acoustic device that is adapted to activate (ii) a cooperating serum that can enhance infusion of agents or active elements of a topical composition into skin, lips or scalp for cosmetic or rejuvenation purposes or other therapeutic purposes.

The present disclosure includes a hand-held acoustic device with at least one ultrasound transducer that applies acoustic energy to a serum carrying micro-vesicles with an interior gas bubble, wherein the acoustic energy causes stable cavitation or inertial cavitation of the micro-vesicles in the serum to apply mechanical forces to a targeted surface of lips or skin which loosens paracellular junctions in the targeted surface to permit penetration of agents and active elements into the subject's lips or skin.

In general, a skin care composition or serum corresponding to the invention comprises a dermatologically acceptable carrier that carries an effective amount of acoustically responsive micro-vesicles that are adapted to oscillate in response to acoustic energy from an acoustic transducer at a frequency range of 0.25 MHz to 5 MHz.

A method corresponding to the invention comprises using acoustic waves to isolate a topical serum carrying acoustically responsive micro-vesicles to apply mechanical forces to a targeted surface of skin or lips of a subject to thereby loosen paracellular junctions.

It will be understood that other objects and purposes of the invention, and variations thereof, will be apparent upon reading the following specification and inspecting the accompanying drawings. These and other features, aspects, and advantages of the present invention will become better understood with reference to the following drawings.

### BRIEF DESCRIPTION OF THE DRAWINGS

FIG. 1 is a plan view of an embodiment of an applicator corresponding to the invention adapted for enhancing fluid penetration into a subject's lips or skin, where the applicator carries an ultrasound transducer, a battery, and a flowable cosmetic composition, wherein the ultrasound transducer is adapted for applying acoustic energy to micro-vesicles carrying a gas, wherein the composition is applied to a targeted surface of the subject's lips or skin.

FIG. 2 is another view of the applicator of FIG. 1 with its component parts separated.

FIG. 3 is an enlarged view of a distal portion of the applicator tip of FIG. 1, showing an ultrasound transducer with stacked piezoelectric elements and the electrical connections to the battery.

FIG. 4 is a schematic view of a distal portion of the applicator of FIG. 1, showing its shape, dimensions, and method of use for treating the subject's lips.

FIG. 5 is an enlarged sectional view of the distal tip of the applicator taken along line 5-5 of FIG. 3 and shows the piezoelectric elements of the transducer positioned against the subject's lips, with the micro-vesicle composition applied to and captured between the distal tip and the targeted surface of the subject's lips.

FIG. 6 is a schematic view of the transducer and piezoelectric elements, micro-vesicle composition, and targeted surface of the subject's lips of FIG. 5, further showing actuation of the transducer which causes oscillation or cavitation of the micro-vesicles, which, in turn, loosens the paracellular junctions to allow the elements of the composition to penetrate layers of the tissue.

FIG. 7 is a schematic view of a variation of a micro-vesicle of the composition of FIGS. 5 and 6 showing a polymeric shell, an inert gas volume therein, and various manners in which components of the topical composition may be coupled to such a micro-vesicle.

FIG. 8A is a schematic view of the anatomical layers of a human subject's lips showing 4-5 stratum corneum layers.

FIG. 8B is a schematic view of the anatomical layers of a human subject's skin showing 15-20 stratum corneum layers.

FIG. 9 is a side view of a distal end of another variation of applicator and ultrasound transducer without an inflow channel for serum application.

FIG. 10 is a side view of a distal end of another variation of applicator with a plurality of ultrasound transducers around a serum inflow channel.

FIG. 11 is a side view of another distal end of an applicator with multiple serum inflow channels around an ultrasound transducer.

FIG. 12 is a view of skin treatment system including a handpiece with a distal tip carrying an ultrasound transducer, a fluid chamber within the handpiece, and a console with a negative pressure source coupled to the handpiece and transducer.

FIG. 13 is a view of the handpiece of FIG. 12 with a handle component de-mated from a transducer component.

FIG. 14 is a partly sectional view of the distal tip of the handpiece of FIGS. 12 and 13, showing fluid inflow pathways.

FIG. 15 is another cut-away view of a distal tip similar to that of FIG. 14, showing fluid inflow pathways.

FIG. 16 is another cut-away view of a variation of a distal tip similar to that of FIGS. 14 and 15 configured with flow channels in the handle component showing fluid inflow pathways.

FIG. 17 is another cut-away view of a variation of a distal tip similar to that of FIG. 16 configured with flow channels in the sides of the transducer showing fluid inflow pathways.

FIG. 18 is a view of a variation of a skin treatment system with a handpiece having a distal tip carrying an ultrasound transducer with a remote fluid source coupled to the handpiece.

FIG. 19 is an enlarged schematic view of a distal portion of the handpiece and distal tip of FIG. 18.

FIG. 20 is an end view of the distal tip and transducer of FIG. 19.

FIG. 21 is a view of a variation of a distal tip and transducer wherein the surface of the transducer is configured with a recess for capturing fluid during use, with the recess comprising a spiral groove.

FIG. 22 is a greatly enlarged schematic view of the transducer of FIG. 21, the microbubble composition, and the targeted skin further showing actuation of the transducer, which causes oscillation or cavitation of the microbubble captured in the recess, which, in turn, loosens the paracellular junctions to allow the active elements of the composition to penetrate the targeted tissue.

FIG. 23 is a variation of a distal tip and transducer with the transducer surface configured with recesses comprising a plurality of pockets or dimples.

FIG. 24 is another variation of a distal tip and transducer with the transducer surface configured with recesses comprising intertwined spiral grooves.

FIG. 25 is another variation of a distal tip and transducer configured with two maze-like recesses.

FIG. 26A is another variation of a distal tip and transducer configured with a plurality of recesses comprising grooves between an inflow outlet and outflow port inside opposing lip portions of the distal tip.

FIG. 26B is a schematic illustration of shape, depth, and width of an exemplary recess in a distal tip and transducer.

FIG. 26C is a schematic illustration of another shape of a recess in a distal tip and transducer.

FIG. 26D is a schematic illustration of another shape of a recess in a distal tip and transducer.

FIG. 26E is a schematic illustration of another shape of a recess in a distal tip and transducer with an edge for manipulating targeted tissue.

FIG. 27 is a view of a variation of a skin treatment system with a handpiece having a distal tip carrying an ultrasound transducer wherein the system allows selection of fluid flows from an interior chamber in the handpiece or fluid flows from a remote fluid source.

FIG. 28 is an enlarged elevational view of a distal portion of the handpiece of FIG. 27.

FIG. 29 is an enlarged perspective view of the distal portion of the handpiece of FIG. 28.

FIG. 30 is another variation of a distal tip of a handpiece and transducer wherein the inflow and outflow channels are both disposed in longitudinal features in a detachable handle portion, which mate with grooves in the transducer and allows for simplified cleaning of the transducer.

FIG. 31 is a view of the distal tip of FIG. 30 with the components de-mated.

### DETAILED DESCRIPTION OF THE INVENTION

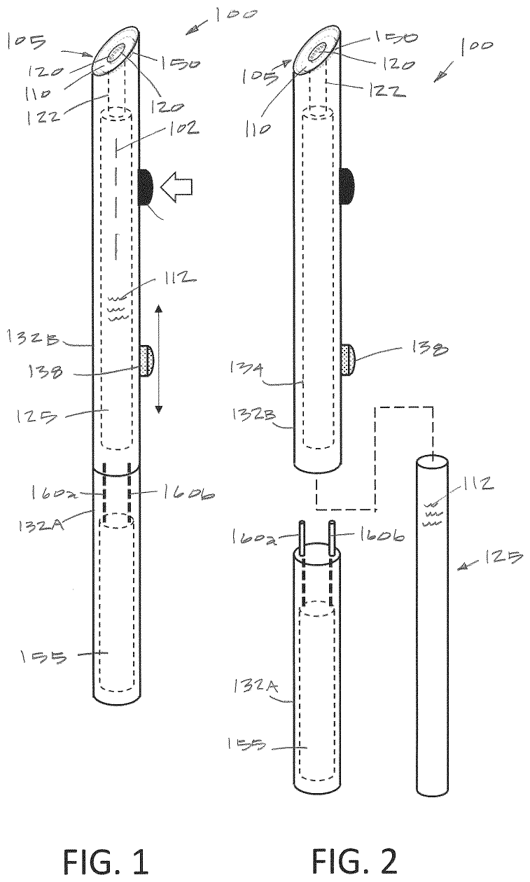

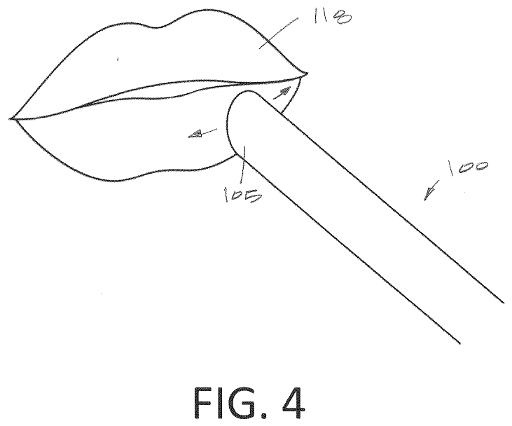

FIGS. 1, 2, and 3 illustrate a system for treating a subject's lips or skin, which comprises a hand-held treatment device or applicator 100 having a longitudinal axis 102 and a distal applicator tip 105 that carries at least one ultrasound transducer 110 adapted for isonation of a serum or skin care composition 112 and a targeted surface 114 of lips or skin (FIG. 4) to enhance fluid absorption and penetration into or through surface layers of such a targeted surface. The applicator 100 is suited for gripping with a subject's fingers for movement over the targeted tissue surface.

In a variation, the distal applicator tip 105 is angled relative to the axis 102 of the applicator for contacting a subject's lips 118, as shown in FIG. 4. The distal tip 105 is typically round or oval, but other shapes are possible. In the variation shown in FIGS. 1-3, the distal tip 105 has a central port 120 and inflow channel 122 communicating with a cartridge 125 that carries the serum or skin care composition 112 for applying to the subject's lips or skin. As can be seen FIG. 2, the applicator 100 has a proximal portion 132A and a distal portion 132B that can be separated from one another to insert the cartridge 125. The thin wall of cartridge 125 is adapted for insertion into a receiving space 134 in the distal portion 132B of the applicator 100. The serum 112 in the applicator can be pushed or pumped from the cartridge to the distal tip 105 with an actuator 138 in the applicator 100. The actuator 138 can slide to impinge upon a flexible-walled cartridge or otherwise can be adapted to pressurize a chamber that carries the serum 112 to cause a flow of the serum distally and outwardly through port 120. In another variation, the cartridge can comprise thin-wall sac or bag that carries the serum. In another variation, the actuator 138 can be coupled to a ratchet mechanism for advancing a plunger in the cartridge. As can be understood from FIG. 1, the actuator 138 is adapted to pump small amounts of serum through the port 120 to be applied to the subject's lips.

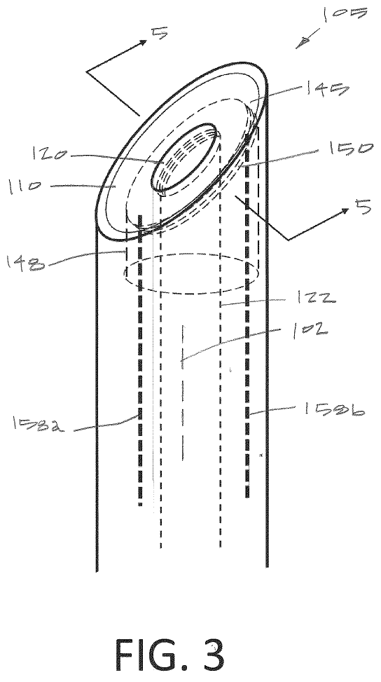

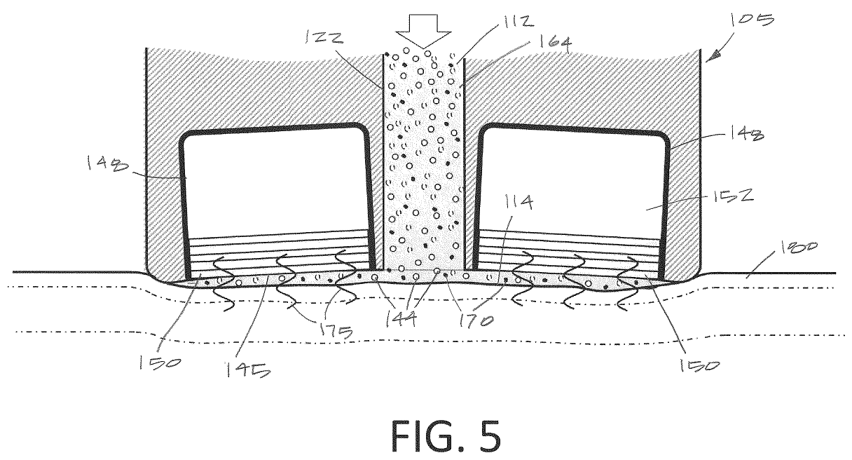

In FIGS. 1 and 3, it can be seen that the distal tip 105 carries an ultrasound transducer 110 configured for applying acoustic energy to the serum 112 and micro-vesicles or microbubbles 144 therein (FIG. 5) as further described below, when the serum 112 is captured between a tissue contacting surface 145 of the distal tip 105 and the subject's lips 118 (FIG. 5). In a variation, the ultrasound transducer 110 comprises a housing 148 carrying a single piezoelectric element or a plurality of stacked piezoelectric elements 150, with stacked piezoelectric elements shown in FIGS. 3 and 5. The transducer 110 carries a backing or damping element 152 adjacent to the proximal-facing surface of the piezoelectric elements 150 as is known in the art (FIG. 5). The transducer 110 also may carry an acoustic lens and impedance matching layer (not shown) distal to the piezoelectric elements 150 for focusing acoustic waves. In a variation shown in FIGS. 1-3 and 5, the transducer 110 is configured with a central opening, which provides the inflow port 120 for the serum 112. FIGS. 1 and 2 further show that the applicator 100 carries a battery 155 in its proximal portion 132A for energizing the piezoelectric elements 150 of the transducer 110. In FIG. 3, it can be seen that electrical leads 158a and 158b are coupled to the piezoelectric elements 150 and extend to the battery 155. FIG. 2 shows the proximal portion 132A and distal portion 132B of the applicator 100 separated from one another. The proximal portion 132A in this variation has projecting electrical connectors 160a and 160b that connect with cooperating features (not shown) in the distal applicator portion 132B and electrical leads 158a and 158b that are coupled to the piezoelectric elements 150. The battery 155 may be recharged as is known in the art by inductive recharging with a cooperating stand (not shown).

FIG. 2 further shows the cartridge 125 carrying the serum 112 separated from the applicator 100 and its components. In a variation, a distal cap can be provided to cover the distal tip 105 when not in use (not shown). Such a distal cap typically would be configured with a projecting portion that would fit into the port 120 to seal the port (not shown).

In a variation shown in FIG. 3, the tissue-contacting surface 145 of distal tip 105 is concave, and the surface of the piezoelectric elements 150 may be angled toward the central axis 102 of the distal tip 105. In other variations shown below, the distal tip 105 may carry a plurality of 2 to 20 piezoelectric elements that may be angled toward one another to focus acoustic waves. Such piezoelectric elements may be singular or stacked as described above to increase amplitude of the movement of the distal surface of such piezoelectric elements 150. It should be appreciated that the piezoelectric elements 150 or acoustic lens (not shown) can have a flat shape, concave shape, or any other suitable shape as is known in the art.

FIG. 4 is a schematic view of a distal region of the applicator 100 of FIG. 1, showing its shape, dimensions, and method of use for treating the subject's lips 118. Referring to FIGS. 1 and 3, the applicator 100 can have any cross-sectional dimension about axis 102 and a shape that is suited for gripping with human fingers. Typically, the applicator 100 has a diameter or cross-section ranging from about 5 mm to 20 mm and often has a cross-section ranging from 10 mm to 15 mm. The components of the applicator 100 of FIGS. 1 to 3 can be fabricated of a molded plastic, metal, a combination of plastic and metal, or other suitable materials. In a variation, the applicator 100 can comprise a transparent or translucent plastic material to allow for viewing of the volume of serum 112 in the cartridge 125 (FIG. 1).

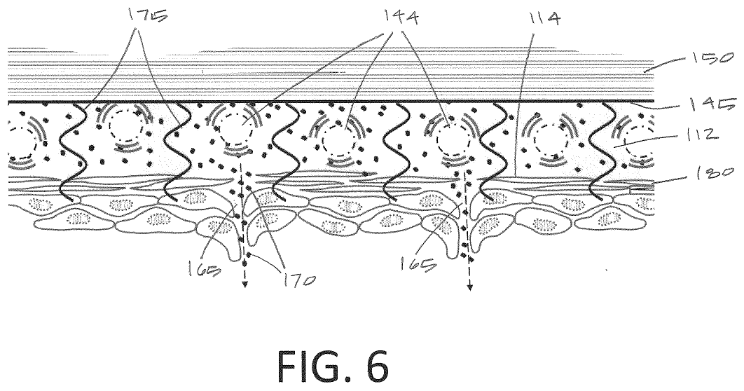

Now, turning to FIGS. 5 and 6, the topical composition or serum 112 for lip or skin care is shown in an interface with a targeted surface 114 of the subject's lips 118. The serum 112 comprises a dermatologically acceptable carrier 164 that carries an effective amount of acoustically responsive microbubbles or micro-vesicles 144 that are adapted to oscillate in response to acoustic energy from the transducer 110 at a frequency range of 0.25 MHz to 5 MHz. The acoustic waves 175 or energy cause stable cavitation or inertial cavitation of the micro-vesicles 144 in the serum 112, which, in turn, applies mechanical forces to the targeted surface 114. Such mechanical forces include forces caused by shock waves or microstreaming from the cavitation, which then loosens paracellular junctions 165 to cause permeabilization of the targeted surface 114 or stratum corneum 180 to permit agents or active elements 170 to penetrate the layers of the stratum corneum 180 (FIG. 6).

The serum 112 comprises a liquid, gel, or paste, and distal tip 105 is translated over the targeted surface 114 while at the same time, additional serum 112 can be pumped into the interface with the targeted surface 114 from the cartridge 125. Acoustic waves 175 are shown propagating through the serum 112 and lips 118.

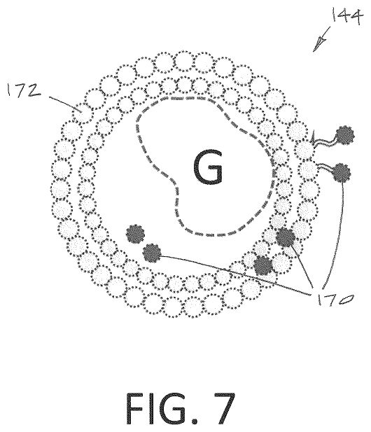

As depicted schematically in FIG. 7, the micro-vesicles 144 have a polymeric shell structure with a single shell 172 or multiple shell layers. An inert gas G is carried in the core of the shell or shell layers. In a variation, the micro-vesicles can comprise albumin shells 172 around an inert gas, such as air or perfluoropropane. The micro-vesicles 144 can be fabricated as is known in the art of making ultrasound contrast agents for intravenous use. (See Yusefil, H. and Helfield, B.; “Ultrasound Contrast Imaging: Fundamentals and Emerging Technology”, Front. Phys. 17; February 2022). The micro-vesicles can range in size from 400 nm to 50 microns and typically can be from 5 microns to 50 microns. The effective amount of such acoustically responsive micro-vesicles 144 is between 0.10% and 10% by weight of the serum 112. In FIG. 7, it can be seen that the agents or active elements 170 can be carried in the interior of a shell 172 or shells or intermediate different layers of multiple shells 172, or coupled to the exterior of a shell of the micro-vesicle 165. Also, the agents or active elements 170 may be floating in the carrier 164 and not coupled to micro-vesicles 144. Further, multiple different agents or active elements 170 can be coupled to different layers of the micro-vesicle shell 172, which can result in different times of release to interface with a targeted surface 114. The carrier can comprise humectants, emollients, and occlusives, as are known in the art.

The serum 112 or topical composition includes agents or active elements 170 for plumping tissue, which includes an effective amount of a capsicum extract or resin from capsicum annuum or capsicum frutescens. Additionally or, alternatively, the serum 112 can include an effective amount of at least one of benzyl nicotinate and methyl nicotinate.

The carrier 164 of the topical composition or serum 112 can include agents or active elements 170 selected from the group of water, hyaluronic acid or derivatives, fractions or fragments thereof, glycerine, ectoin, niacinamide, propylene glycol, hydrogenated lecithin, capric triglyceride, cholesterol, linoleamidopropyl Pg-dimonium chloride phosphate, tocopheryl acetate, tocopherol, vitamin C, mentha piperita oil, glyceryl stearate, dimethicone, synthetic beeswax, cetyl alcohol, oleth-2, caprylyl glycol, caprylhydroxamic acid, paraffinum liquidum, persea gratissima oil, mica, simmondsia chinensis seed oil, ethylhexyl palmitate, tribehenin, sorbitan isostearate, palmitoyl oligopeptide, tocopheryl acetate, pentaerythrityl tetraisostearate, silica dimethyl silylate, sodium chondroitin sulfate, atelocollagen, phenoxyethanol, lactic acid, mandelic acid, and glycolic acid.

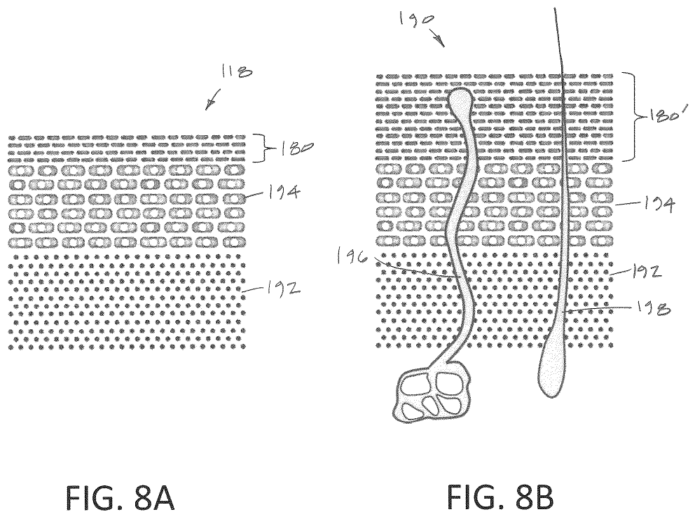

While the method of oscillating or cavitating the micro-vesicles 144 in a serum 112 to treat lips 118 is shown in FIGS. 5 and 6, the method is also suitable for treating facial skin as well as skin of other portions of the human body. FIGS. 8A and 8B illustrate the difference between the anatomical structure of lips 118 (FIG. 8A) versus facial skin 190 or other skin (FIG. 8B). Although all skin is composed of three main layers, the same three layers are different on a subject's lips 118. The deepest layer is the dermis 192, and the next most superficial layer is the living epidermis 194, which is the layer responsible for producing new cells as well as melanocytes in facial and other skin, the cells that are responsible for producing melanin, which gives skin its color and helps protect from UV rays. The surface layer is the stratum corneum, which is a thin protective layer essentially composed of overlapping dead skin cells.). As can be seen in FIG. 8A, the superficial stratum corneum layer 180 of a subject's lips 118 is about 3-4 layers thick, which is very thin compared to typical facial skin 190 of FIG. 8B, which generally has 15 to 20 layers in its stratum corneum 180′. Facial skin 190 also carries portions of sweat glands 196 and hair follicles 198 (FIG. 8B).

Thus, when using the methods of the invention to treat facial or other skin 190, it is preferable to exfoliate and remove surface layers of the stratum corneum 180′ of FIG. 8B in advance of using the ultrasound transducer 110 or transducers to apply acoustic energy. Further, it is preferable to use higher levels of acoustic energy which oscillates or cavitates the micro-vesicles 144 to loosen the paracellular junctions as described above while at the same time, the higher levels of acoustic energy can directly loosen paracellular junctions to thereby permit deeper penetration of agents or active elements 170.

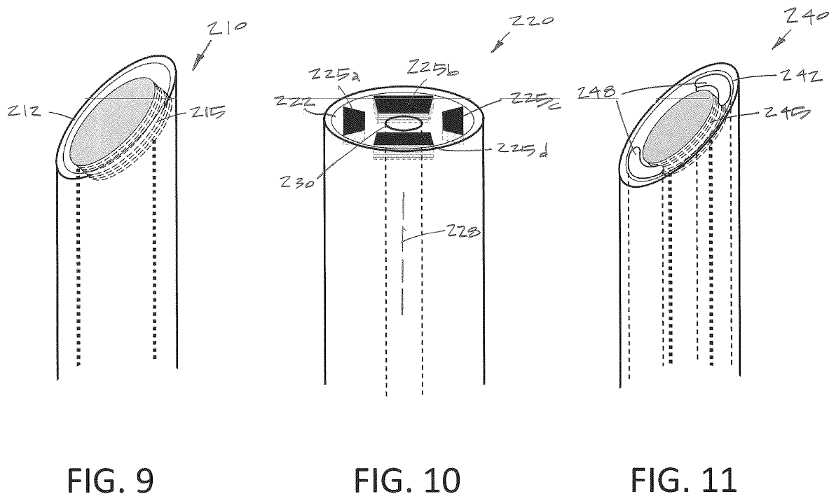

FIG. 9 shows another variation of applicator 210 wherein the distal tip 212 again carries a stacked piezoelectric transducer 215 as described above but without a serum inflow channel for applying serum to a subject's lips. In this variation, the serum 112 can be applied with a swab or lipstick-type applicator (not shown) that is independent of the applicator of FIG. 9. In all other aspects, the transducer 215 of FIG. 9 would operate to loosen paracellular junctions and cause penetration of agents and active elements 170 as described above.

FIG. 10 shows another variation of applicator 220 wherein the distal tip 222 carries a plurality of ultrasound transducers 225a-225d spaced around an axis 228 of the applicator that are configured to focus ultrasound waves toward the axis 228 of the applicator. In this variation, a central inflow channel 230 is shown for applying serum to the subject's lips.

FIG. 11 shows another variation of applicator 240 wherein the distal tip 242 has a central transducer 245 for delivering ultrasound energy, as described above. In this variation, one or more serum inflow channels 248 are positioned outwardly from to the transducer 245.

In general, a method of treating a subject's skin or lips comprises (i) providing a hand-held applicator carrying at least one ultrasound transducer configured to deliver acoustic energy at a distal tip of the applicator; (ii) applying a topical composition or serum carrying acoustically responsive micro-vesicles to a targeted surface of a subject's skin or lips; (iii) positioning the distal tip in contact with the targeted skin or lips and composition; and (iv) actuating the at least one ultrasound transducer at a frequency range of 0.25 MHz to 5 MHz to cause an oscillating response of the micro-vesicles to enhance application of the composition into the targeted surface. The oscillating response of the method causes at least one of stable cavitation and inertial cavitation of the micro-vesicles in the serum. The stable cavitation or inertial cavitation of the micro-vesicles in the serum applies mechanical forces to the targeted surface, which loosens paracellular junctions in the targeted surface to permit penetration of agents and active elements. The application of acoustic energy may be continuous or pulsed. In a variation, the system can be designed to include a flash mode wherein pulses of high acoustic energy can be applied to burst and eliminate substantially all of the gas-filled bubbles rapidly when the serum application is complete.

In another variation, an applicator can carry a resistive heating element that is adapted to warm the serum to a temperature between 39° C. and 41° C. as it is applied to enhance agent penetration. In another variation, the applicator can carry an LED with a wavelength, such as infrared, which can be used to warm the serum and potentially warm the targeted tissue surface 114 to enhance agent penetration.

In another variation, an applicator tip can carry micro-actuators for vibrating the distal tip and skin surface in conjunction with energy applied by an ultrasonic transducer. Such micro-actuators are disclosed in commonly-invented U.S. Pat. No. 10,456,321.

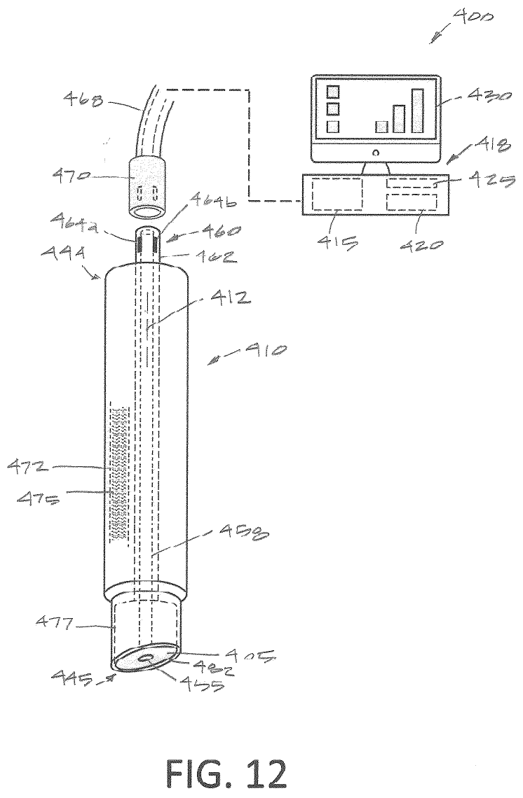

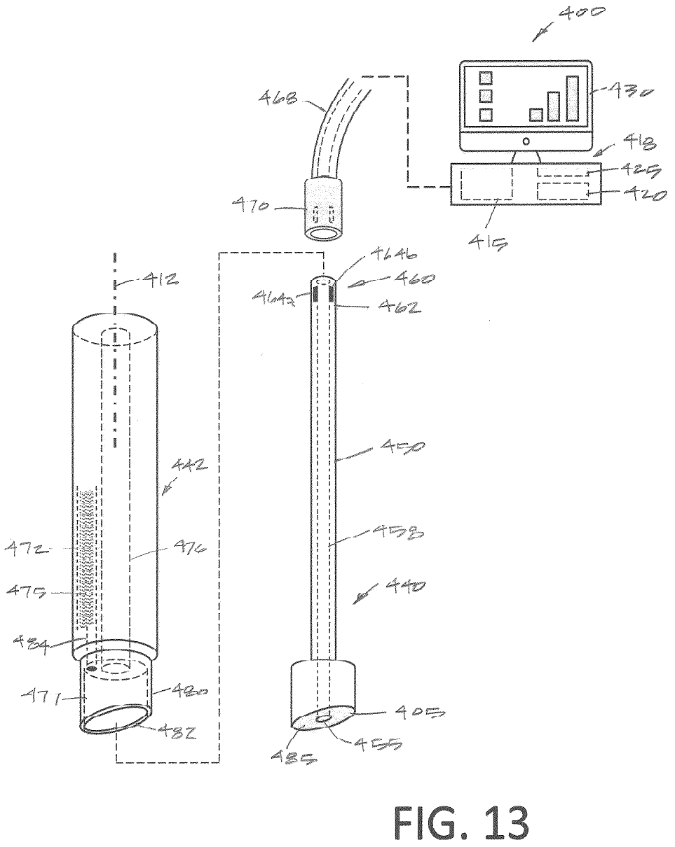

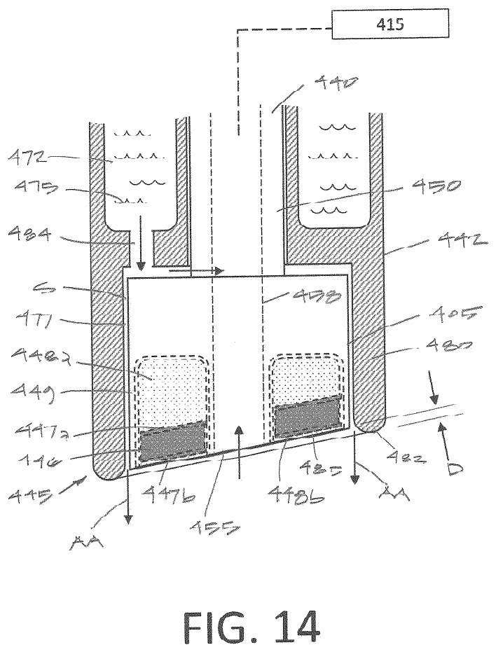

Now, turning to FIGS. 12 and 13, another variation ultrasonic treatment system 400 is shown that is adapted for facial skin treatments and the application of serums to a targeted skin surface while contemporaneously permeabilizing the targeted skin surface for deeper penetration of the serums into the targeted skin. In the variation of FIGS. 12 and 13, an ultrasound transducer 405 or plurality of transducers operates as described above. In this variation, the applicator or handpiece 410 extends about axis 412 and is configured for applying negative pressure to the targeted skin surface from a remote negative pressure source 415 in a console 418, wherein the use such negative pressure will draw a fluid serum from a fluid source than comprises a fluid reservoir in a handpiece assembly or from a remote fluid serum source through flexible tubing as will be described below. In this variation, the transducer 405 is configured for connection to remote electrical source 420 and controller 425 in the console 418 as will be further described below. In a variation, a touchscreen display 430 is provided to adjust operating parameters of the negative pressure source 415 and ultrasound transducer 405.

In FIGS. 12 and 13, the applicator or handpiece or 410 is shown in assembled and disassembled configurations, respectively. As best seen in FIG. 13, the handpiece 410 comprises an assembly of a first component or transducer component 440 and a second handle component 442 that is shaped for gripping by the operator's hand. In the assembled configuration of FIG. 12, the handpiece 410 extends from a proximal end 444 to a tissue-contacting distal tip 445. The ultrasound transducer 405, or plurality of transducers, in the transducer component 440 can be the same as described above in previous variations. In the enlarged view of FIG. 14, it can be seen that the term ultrasound transducer 405, as used in describing this variation, refers to the distal assembly of the transducer component 440 comprising five main components, as known in the art, consisting of a crystal or ceramic clement 446 with piezoelectric properties, electrodes 447a and 447b on opposing faces of the piezoelectric element, a damping block 448a, a matching layer 448b and a housing 449.

In the variation of FIGS. 12 and 13, the transducer component 440 is re-usable and can be cleaned or otherwise sterilized. In this variation, the transducer component 440 that carries the transducer 405 is coupled to an elongated proximal body portion or shaft portion 450 that is configured to extend through the handle component 442 when assembled. As can be seen in FIG. 13, an aspiration port 455 is positioned in the transducer 405 that comprises the open end of an aspiration channel 458 extending through the shaft portion 450 of the transducer component 440. A first connector 460 is provided at the proximal end 462 of the shaft portion 450. The first connector 460 has electrical contacts 464a 464b connected to electrical leads that extend through the shaft portion 450 to energize the transducer 405. A flexible conduit 468 is configured to connect the handpiece 410 to the remote negative pressure source 415, electrical source 420, and controller 425. As can be seen in FIGS. 12 and 13, the distal end of the conduit 468 has a second connector 470 adapted to couple to the first connector 460 of the handpiece 410.

Still referring to FIGS. 12 and 13, the second handle component 442 is typically a molded plastic and is configured with a chamber 472 that carries a treatment fluid or serum 475. In this variation, the chamber 472 has an annular configuration around a central passageway 476 (FIG. 13) therein that receives the shaft portion 450 of the transducer component 440. It should be appreciated that the handpiece 410 can be designed in various configurations that provide a first transducer component that is re-usable and a second body or handle component that carries a fluid chamber or reservoir, with such a second body component configured for single use or multiple use. It should be appreciated that the scope of the invention includes a handpiece with a unitary body that carries a transducer for coupling with a remote fluid source, wherein such a unitary body is cleanable or sterilizable. Other handpiece variations fall within the scope of the invention that include a transducer body configured to receive a fluid-filled cartridge or handpieces as described below that are configured for selecting a serum flow from an interior chamber and/or from a remote reservoir.

As can be seen in FIGS. 13 and 14, the distal end of the handle component 442 is configured with a recess 477 that is dimensioned to receive the transducer 405. The recess 477 is surrounded by a thin wall 480 with a distal periphery that forms a lip 482 around the outer surface of the transducer 405 when the handpiece components are assembled. As can be seen in FIGS. 13 and 14, at least one flow channel 484 extends between the chamber 472 and the recess 477 in the handle component. In FIG. 14, arrows AA depict the inflow of serum 475 through an annular space S between the inner surface of recess 477 and lateral surfaces of the transducer 405. The components are dimensioned to allow fluid flow through the annular space S.

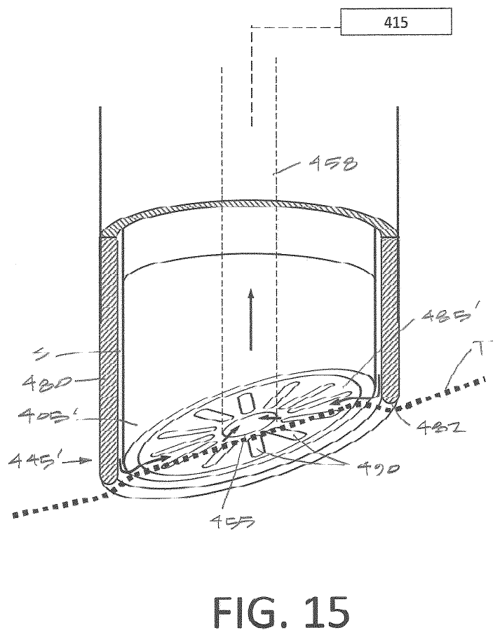

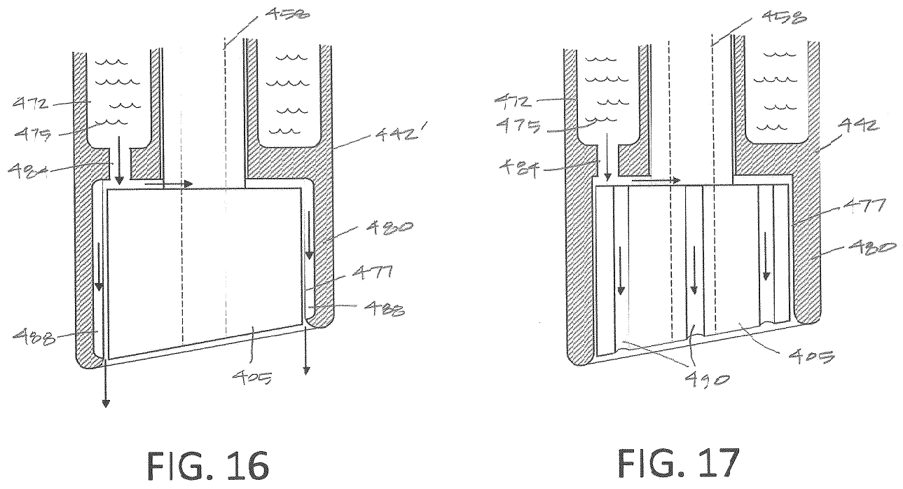

Still referring to FIG. 14, the distal-facing surface 485 of the transducer can be angled or transverse to axis 412 of the handpiece 410. In any such configuration, as best seen in FIG. 14, the distal periphery of wall 480 that forms lip 482 can extend a distance D from 0.2 mm to 2.0 mm distally beyond the outer edge of the transducer 405. As described previously, the distal-facing surface 485 of the transducer can be flat or concave. In use, the distal tip 445 of handpiece 410 is pressed into an interface with a targeted skin surface T (see FIG. 16), wherein the lip 482 is thus adapted to form a seal around the perimeter of distal tip 445, which in turn causes negative pressure in the interface with targeted tissue to draw serum 475 from the chamber 472 into the annular space S between and around the lateral surfaces of the transducer 405. In FIG. 14, arrows AA depict the inflow of serum 475 through the annular space S without targeted skin shown. FIG. 15 is a similar distal tip 445′ of the handpiece with inflow arrows AA showing the serum inflows directed across the interface between the transducer 405 and targeted skin T. Thus. The negative pressure causes a circulating flow of serum 475 across the targeted skin from the inflow channel 484 to the outflow port 455 in the transducer 405 and such a circulating flow only occurs when the distal tip 445 is in contact with the targeted skin T.

To use the system 400, the operator typically can select a negative pressure on the touchscreen 430, as shown in FIGS. 12 and 13. The operator also can select an operating mode on the touchscreen 430 among various energy levels and duty cycles for transducer. In a variation, the controller 425 can be connected to a sensor in the negative pressure source that can sense pressure changes in the outflow channel 458, which can indicate that the distal tip 445 is sealed against the targeted tissue T or that the distal tip 445 is not in contact with tissue. The controller 425 then can be configured to activate the transducer 405 only when the distal tip 445 is in contact with targeted tissue. In another variation, the periphery of the distal tip 445 can carry a contact sensor to determine tissue contact to thereby activate the transducer 405. A capacitance sensor or impedance sensor can be used for this purpose. In another variation, the handpiece 410 can carry a finger actuated switch for activating the transducer.

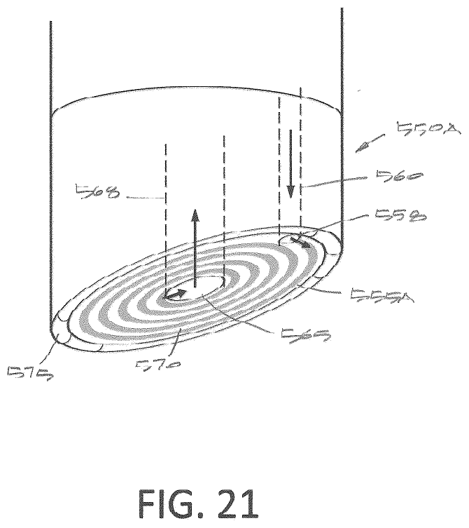

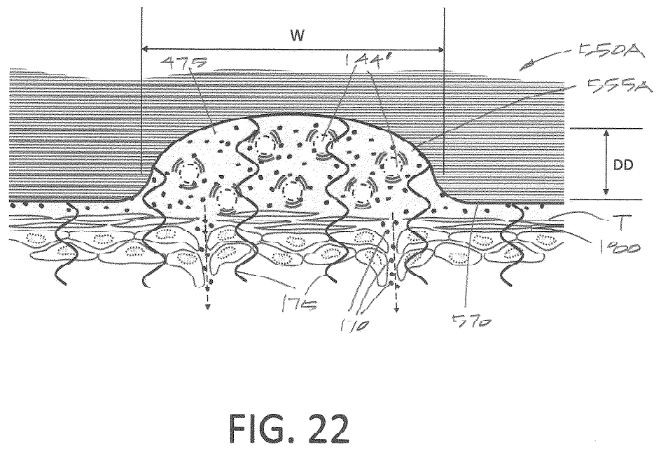

FIG. 15 is a cut-away view of a distal tip 445′ of a handpiece that is similar to that of FIG. 14 with a different transducer variation. In FIG. 15, it can be seen that the distal surface 485′ of ultrasonic transducer 405′ has a non-smooth configuration, which differs from ultrasonic transducers known in the art. In a variation, the transducer surface 485′ that emits ultrasonic energy toward tissue has recesses 490 that are adapted to capture fluid or serum 475 in the interface between the surface 485′ and the targeted skin T. As the distal tip 445′ is translated over the targeted skin, serum 475 can be momentarily captured in the recesses 490, wherein the ultrasonic energy then causes cavitation of the gas-filled microbubbles 144 in the recesses 490 which in turn creates pressure waves to cause the active components 170 of the serum 475 to penetrate the targeted skin, as shown in FIG. 22 and further described below. In the variation of FIG. 15, a plurality of recesses 490 is shown that extend radially around the surface 485′ of the transducer 405. Other variations of such fluid-capturing recesses 490 are shown in FIGS. 21-26 and FIGS. 30-31.

Now, turning to FIG. 16, another variation of a handpiece component 442′ is shown that is similar to the handpiece of FIGS. 14-15, except that the thin wall 480 around the recess 477 that receives the transducer 405 is configured with at least one longitudinal groove 488 to direct a flow of fluid from chamber 472 and inflow channel 484 to the interface between the transducer surface 485 and targeted skin. This variation allows for a free flow of serum 475 from chamber 472. FIG. 17 shows another variation that is similar to the device and handle component 442 of FIGS. 15-16, except that longitudinal flow channels 490 are formed in the outer surfaces of the transducer 405 to provide fluid flow pathways for introducing serum into the interface between the transducer surface 485 and targeted skin.

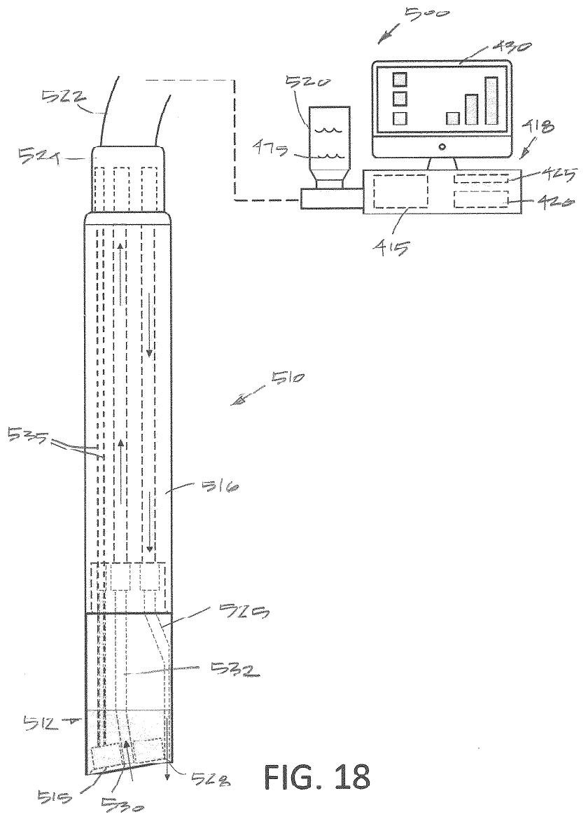

FIGS. 18-20 illustrate another variation of system 500 and handpiece 510 that differs from the system of FIGS. 12 and 13. As can be seen in FIGS. 18 and 19, the handpiece 510 comprises a first component, 512 carrying the ultrasound transducer 515, and a second component comprising a handle member 516. In this variation, the handpiece 510 does not carry an interior chamber for carrying a fluid serum. Instead, at least one fluid serum source 520 is positioned remote from the handpiece 510, for example, positioned on or proximate to the console 418 carrying the negative pressure source 415, electrical source 420, and controller 425. Thus, in the system 500 of FIG. 18, a conduit 522 with connector 524 couples the handpiece 510 with the console 418 and serum source 520. Referring to FIG. 18, the first and second components, 512 516, are configured with a serum inflow channel 525 and flow outlet 528 in the transducer 515. A fluid outflow port 530 and outflow channel 532 in the handpiece 510 communicate with the negative pressure source 415. Also, electrical leads 535 extend through the assembled handpiece 510 to the transducer 515, as described previously. In FIG. 18, the conduit 522 and connector 524 are adapted to connect to electrical leads 535, inflow channel 525, and outflow channel 532 in the handpiece.

FIG. 19 is an enlarged view of the first component 512 of FIG. 18 de-mated from the distal end of the handle member 516. It can be seen that male-female connectors are configured to couple the inflow channel 525 and outflow channel 532 as well as couple the electrical leads 535 to the transducer 515. In this variation, the first component, 512, is designed for multiple use and can be easily cleaned since the inflow and outflow channels are short. The handle component 516 can be adapted for single use or multiple use. In another variation, a handle component 516 and transducer 515 can comprise a single unitary body, but it may be less than optimal due to the need to clean or sterilize elongated inflow and outflow channels in such an embodiment.

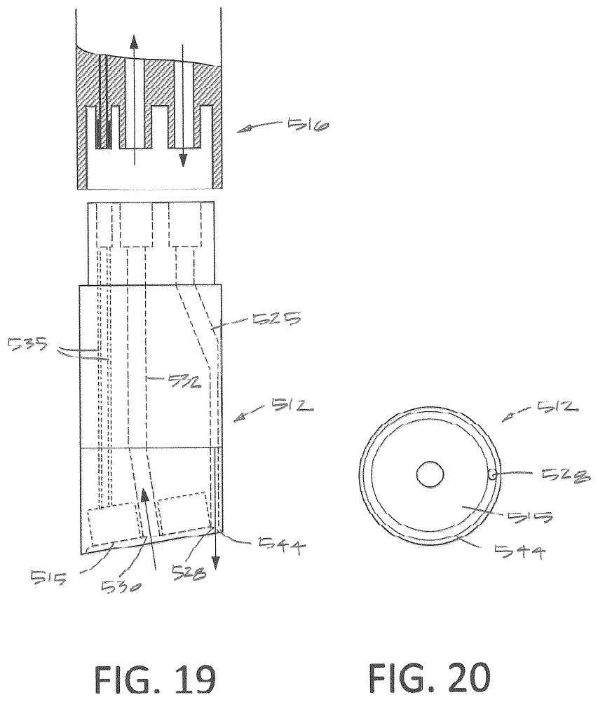

Referring to FIGS. 18 and 19, the inflow channel 525 and outlet 528 is positioned in the transducer 515 rather than about an outer surface of the transducer as in the variation of FIGS. 12-14. In FIGS. 19 and 20, it can be seen that the outlet 528 is inside a lip 544 at the distal periphery of the transducer 515. The distal surface 545 of the transducer 515 again is recessed inward of the lip 544. In other words, the transducer surface 545 again has a recessed region inward of lip 544, which allows the lip to seal against targeted tissue T as shown above in FIG. 15. In all other respects, the variation FIGS. 18-20 operated as described previously to permeabilize skin surface and contemporaneously cause fluid serum 475 to penetrate the target skin surface.

Now turning to FIGS. 21 to 26, other variations of ultrasonic transducers 550A-550D are shown, wherein the distal surface of each variation carries a different form of one or more recesses to momentarily capture and/or direct the flow of serum 475 during a treatment procedure. In FIG. 21, it can be seen that transducer 550A is configured with a spiral shaped recess 555A or groove in which serum would potentially flow from the outlet 558 of inflow channel 560 toward an outflow port 565 communicating with an outflow channel 568 and the negative pressure source as described above. The spiraling flow of serum in recess 555A has the effect of maintaining the serum in contact with targeted tissue T (see FIG. 15) for an extended time interval, during which the serum 475 is cavitated which is advantageous.

FIG. 22 is a greatly enlarged schematic view of an interface between the surface 570 of transducer 550A of FIG. 21 and a targeted skin surface T, for example, a subject's facial skin. The surface 570 of the transducer and recess 555A therein captures the serum 475 that again carries an effective amount of acoustically responsive elements, which can comprise microbubbles 144 as described above or nanoparticles 144′ illustrated in FIG. 22. The microbubbles 144 or nanoparticles 144′ are adapted to respond to acoustic energy waves 175 from the transducer 550A at a frequency range of 0.10 MHz to 20 MHz. As described above with reference to FIGS. 5 and 6, the acoustic waves 175 or energy cause stable cavitation or inertial cavitation of the microbubbles 144 in the serum 475, which, in turn, applies mechanical forces or pressure to the targeted skin T. In a variation, the nanoparticles 144″ can comprise nanoscale, phase-change particles that are known as phase-change contrast agents (PCCAs). Such particles can be phase-transitioned into highly echogenic microbubbles by means of ultrasound energy. The application of ultrasound energy causes the phenomenon of acoustic droplet vaporization to produce bubbles and has been investigated for both imaging and therapeutic applications. In a variation, the acoustic effects result in pressure waves or mechanical forces at a nano-scale or micro-scale that can permeabilize anatomical and/or functional membranes such as a subject's skin, FIG. 22 schematically illustrates mechanical forces that loosen paracellular junctions 165 to cause permeabilization of the targeted skin T to cause agents or active elements 170 to penetrate the layers of the stratum corneum 180 similar to that of FIG. 6. In this variation, the recess 555A can capture a volume of serum 475 subject to acoustic energy more readily than a flat transducer surface that can displace serum from the skin interface. The use of acoustically responsive nanoparticles, and types of nanoparticles potentially useful for skin permeabilization, are described in S. Zullino, et al., From Micro-to Nano-Multifunctional Theranostic Platform: Effective Ultrasound Imaging Is Not Just a Matter of Scale; Molecular Imaging; Volume 17, January-December 2018, and T. Matsunaga et al., Phase-Change Nanoparticles Using Highly Volatile Perfluorocarbons: Toward a Platform for Extravascular Ultrasound Imaging; Theranostics, December 2012:1185-98.

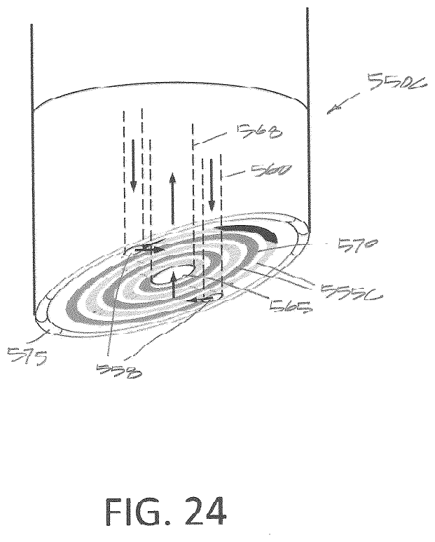

It should be appreciated that any recess in a transducer surface 570 can be similar in width W and depth DD as recess 555A in FIGS. 21 and 22, wherein such recesses can comprise one or more grooves, dimples, pockets, channels, or the like, wherein the channels can comprise spiral channels, radial channels, concentric channels, interrupted channels, or maze-like channels with variations shown FIGS. 15, 21, and FIGS. 23-26. The recess can have a width W ranging from 0.01 mm to 5.0 mm and a depth DD of 0.01 mm to 3.0 mm. The surface 570 of any transducer is typically recessed with a peripheral lip 575 (FIG. 21), and the fluid outlet 558 is provided inside the lip 575. In some variations, there may be a plurality of flow outlets 558, as shown in the variation of FIG. 24. In other variations, the fluid outflow port 565 or ports and outflow channel 568 may be at a periphery of the transducer surface 570 and the at least one flow outlet 558 in the center of the transducer. In another variation, the inflow outlet 558 and the outflow port can be in opposing peripheral portions of the transducer surface 570.

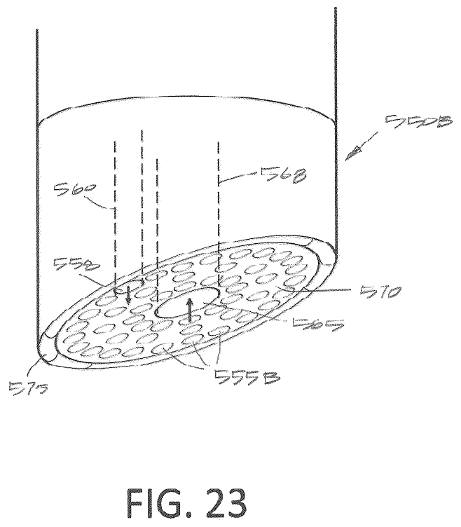

FIG. 23 illustrates a variation of transducer 550B with a plurality of recesses or pockets 555B in the transducer surface 570 for capturing serum. The inflow outlet 558 is at the periphery of the transducer. This variation again has a central outflow port 565 that communicates with the outflow channel 568 and negative pressure source.

FIG. 24 illustrates a variation of transducer 550C with surface 570 configured with intertwining spirals 555C that extend from first and second inflow outlets 558 at the periphery of the transducer. This variation again has central outflow port 565 that communicates with the outflow channel 568.

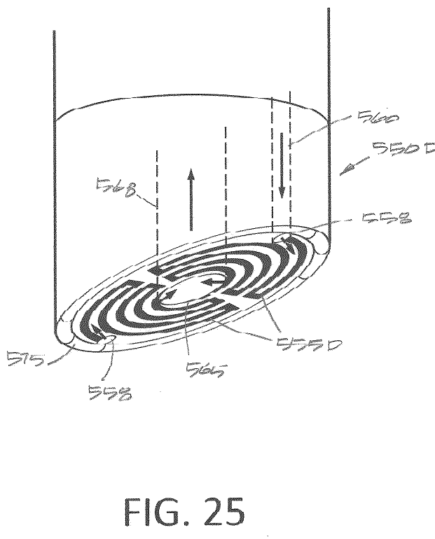

FIG. 25 is another variation of a transducer 550D with maze-like channels 555D that extend from first and second inflow outlets 558 at the periphery of the transducer. This variation again is configured with central outflow port 565 and outflow channel 568.

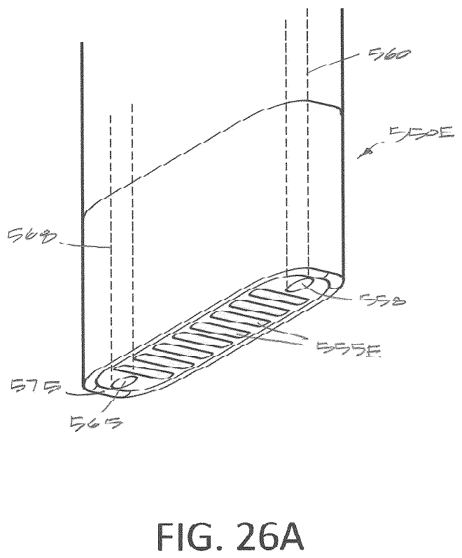

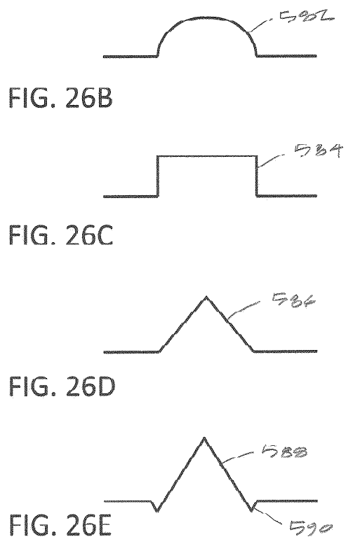

FIG. 26A is another variation of transducer 550E with a single inflow outlet 558 at one position in the periphery of the transducer 550E and a single outflow port 565 at the opposing side of the periphery of the transducer. This variation is configured with a plurality of recesses 555E for capturing serum. This transducer variation has an elongated shape, and it should be appreciated that round, oval, rectangular, and other rounded shapes may be used.

FIGS. 26B to 26E are cross-sectional views of exemplary shapes of recessed in a transducer surface having the width and depth described above. FIG. 26B shows a recess with concave shape 582. FIG. 26C shows a rectangular shaped recess or groove 584. FIG. 26D shows a recess with a triangular shape 586. FIG. 26E shows a recess 580 with an edge feature 590 that is adapted to manipulate, stretch, or exfoliate a targeted skin surface as the tip is moved over a subject's skin.

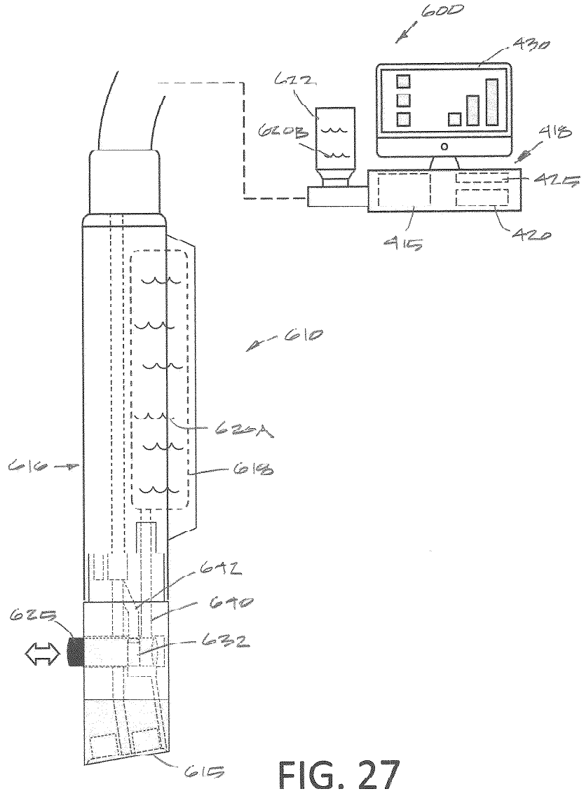

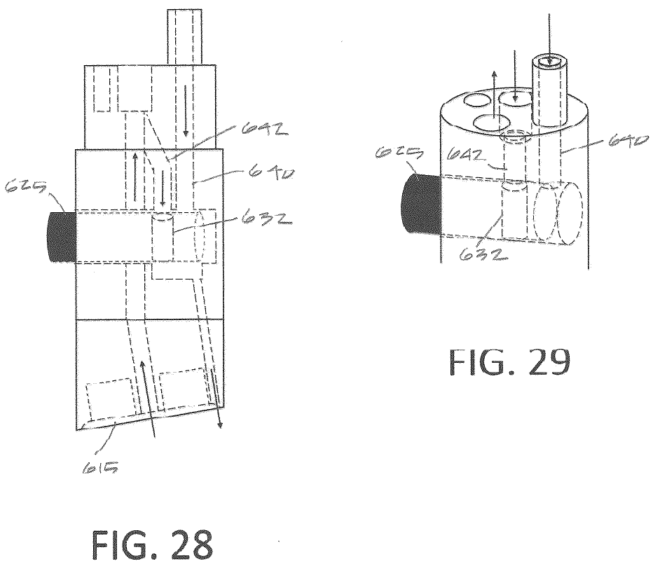

FIG. 27 shows another variation of system 600 and handpiece 610 with transducer 615 and handle component 616 that incorporates features from the variations described above. The handpiece 610 includes an interior chamber 618 that carries a first serum 620A, and the handpiece also is coupled to remote fluid serum reservoir 622 that carries a second serum 620B. In this variation, the operator can use an actuator 625 in the handpiece 610 to select between the serum or fluid in chamber 618 or the serum or fluid in remote reservoir 622. As can be best seen in FIGS. 28 and 29, a spring-loaded actuator 625 can be moved to allow fluid to flow from a first source while preventing fluid flow from the second source as can be understood with the flow channel 632 in the actuator. In FIGS. 28 and 29, it can be understood how the channel 632 in the actuator 625 can be aligned with the inflow channel 640 from the interior chamber 618 or the inflow channel 642 from the remote reservoir 622. The actuator 625 can be spring-loaded, but for convenience, the spring is not shown. In this variation, the transducer 615 is adapted for multiple use, and the handle component 616 may be adapted for single use or multiple use.

In another system variation, the handpiece and an actuator are adapted to mix fluid from first and second fluid sources. For example, the chamber in a handpiece similar to that of FIG. 27 can carry a fluid with microbubbles, which in some variations will comprise microbubbles that have a limited lifetime before degrading. This system then can draw a microbubble composition (fluid or non-fluid) from a chamber in the handpiece to combine with fluid from another source to introducing into the interface with targeted skin. The second source can be a chamber, cartridge in the handpiece or a remote source.

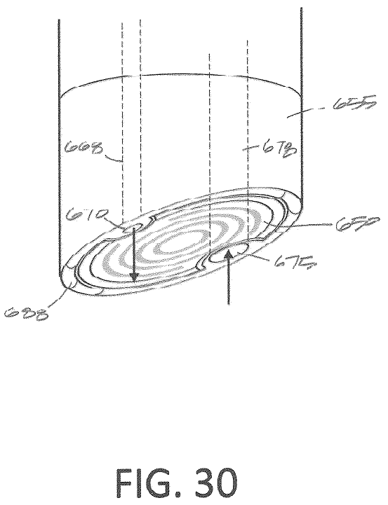

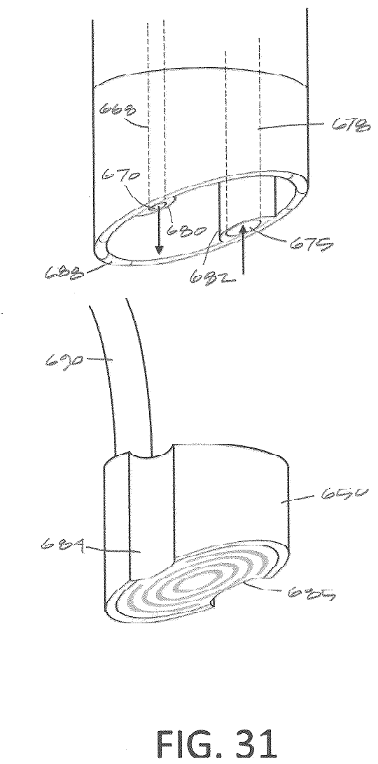

FIGS. 30 and 31 illustrate another variation of handpiece with a transducer 650 and detachable handle portion 655. This variation differs in that the transducer 650 has no interior channels for serum inflows and/or outflows as in the previous variations. Thus, the ultrasound transducer 650 of FIGS. 30 and 31 is easier to clean or sterilize. As can be seen in FIG. 31, the handle portion 655 is configured with the inflow channel 668 and inflow outlet 670. Further, the handle portion 655 is configured with the outflow port 675 and outflow channel 678. The inflow and outflow channels 668 and 678 are carried inwardly, projecting features 680 and 682, respectively, in opposing walls of the handle portion. FIG. 31 further shows that the transducer 650 has longitudinal notches 684 and 685 for receiving the inwardly projecting features 680 and 682. The components are again designed to provide the inflow outlet 670 and outflow port 675 inward of the lip 688 provided by the handle portion. In this variation, the transducer 650 is coupled to an electrical cable 690 with a connector (not shown) that can be inserted through interior passageway 692 in handle portion 655 for coupling to a conduit from the console (FIG. 12). The inflow channel 668 can be coupled to a chamber in the handle portion 655 and/or to a remote serum source as described above.

In another variation, the systems described above can be further adapted to exfoliate skin by different mechanisms. First, in any variations above, the distal lip around a transducer can carry sharp features such as diamond dust to abrade tissue. In an alternative variation, the serum can carry an abrasive powder such as diamond dust, ground walnut shells, or the like to abrade skin as the lip of the distal tip rolls over targeted tissue.

While the invention has been described for delivery of treatment media to a subject's lips and skin largely for cosmetic and rejuvenation purposes, an applicator can also be used for enhancing the delivery of any type of pharmaceuticals through an exfoliated skin surface, such as analgesics, anti-inflammatory drugs and the like.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference as if set forth in its entirety herein.

What is claimed is:

1. A system for treating a skin of a patient using a serum comprising a plurality of microbubbles and a plurality of active elements, the system comprising:
a handpiece comprising a proximal end and a distal end extending along a longitudinal axis;
a distal tip at the distal end of the handpiece, the distal tip configured to deliver the serum;
an ultrasonic transducer in the distal tip;
an outflow port in the distal tip coupled to a remote negative pressure source; and
an inflow port in the distal tip in fluid communication with a source configured to hold the serum, where the inflow port is configured for topical application to the skin;
wherein application of acoustic energy from the ultrasonic transducer causes the plurality of microbubbles in the serum delivered to the skin to oscillate causing cavitation of the plurality of microbubbles causing permeabilization of the skin allowing for penetration of the plurality of active elements in the serum to penetrate the skin.

2. The system of claim 1, wherein the serum is delivered to the distal tip from a serum source remote from the handpiece.

3. The system of claim 1, wherein the serum is carried in the handpiece.

4. The system of claim 1, wherein the distal tip comprises a concave surface.

5. The system of claim 1, wherein the distal tip comprises a plurality of piezoelectric elements angled towards the longitudinal axis.

6. The system of claim 1, wherein the distal tip comprises a plurality of piezoelectric elements in a stacked configuration.

7. The system of claim 1, wherein the distal tip comprises a plurality of ultrasound transducers, wherein the plurality of ultrasound transducers are spaced around the inflow port.

8. The system of claim 1, further comprising one or more serum inflow ports positioned outwardly from the ultrasonic transducer.

9. The system of claim 2, wherein the serum source comprises a first remote source and a second source comprising a cartridge attached to the handpiece.

10. The system of claim 9, further comprising a mechanism in the handpiece for selecting the first remote source or the second source.

11. The system of claim 1, further comprising electrical leads extending from the ultrasonic transducer through the handpiece to a connector configured for connection to an electrical source.

12. The system of claim 1, further comprising an actuator in the handpiece for activating the ultrasonic transducer.

13. The system of claim 1, further comprising a controller operatively connected to the ultrasonic transducer and electrical source, wherein the controller is configured for selection of a plurality of modes of operation of the ultrasonic transducer.

14. The system of claim 1, wherein the distal tip comprises one or more recessed surfaces configured to capture serum.

15. The system of claim 1, wherein the serum comprises a liquid, gel, or paste.

16. A method of treating a targeted skin of a patient, comprising:
providing a hand-held applicator with a distal tip carrying one or more ultrasound transducers configured to deliver acoustic energy to the targeted skin;
introducing a serum carrying a plurality of microbubbles and a plurality of active elements to the targeted skin via an inflow port in the hand-held applicator;
actuating the one or more ultrasound transducers to apply acoustic energy to the serum oscilate the plurality of microbubbles, causing cavitation of the plurality of microbubbles to produce permeabilization of the targeted skin allowing the plurality of active elements in the serum to penetrate the targeted skin.

17. The method of claim 16, wherein the serum is captured in one or more recesses in a surface of the one or more ultrasound transducers during application of acoustic energy.

18. The method of claim 16, wherein the one or more ultrasound transducers are actuated at a frequency range of 0.1 MHz to 10 MHz.

19. A method of treating a targeted skin on a patient, comprising:
providing a hand-held applicator with a distal tip carrying an ultrasound transducer configured to deliver acoustic energy to the targeted skin;
introducing a serum to the targeted skin, the serum carrying a plurality of nanoparticles and a plurality of active elements;
actuating the ultrasound transducer at a frequency range of 0.1 MHz to 10 MHz to apply acoustic energy to the serum, which causes an oscillating response of the plurality of nanoparticles resulting in a phase change of the plurality of nanoparticles, where the phase change of the plurality of nanoparticles causes permeabilization of the targeted skin allowing the plurality of active elements to penetrate the targeted skin.

20. The method of claim 19, wherein the ultrasound transducer is actuated at a frequency range of 0.1 MHz to 10 MHz.
